(19) United States
(12) Patent Application Publication
(10) Pub. No.: US 2024/0395039 A1
(43) Pub. Date: Nov. 28, 2024

(54) SYSTEM AND METHOD FOR SEARCHING AND PRESENTING SURGICAL IMAGES

(71) Applicant: Kaliber Labs Inc., San Francisco, CA (US)

(72) Inventors: Chandra JONELAGADDA, San Francisco, CA (US); Aneesh JONELAGADDA, San Francisco, CA (US); Mark RUIZ, San Francisco, CA (US)

(21) Appl. No.: 18/692,794

(22) PCT Filed: Sep. 15, 2022
(86) PCT No.: PCT/US2022/043723

§ 371 Date: Mar. 15, 2024

Related U.S. Application Data
(60) Provisional application No. 63/244,385, filed on Sep. 15, 2021.
(60) Provisional application No. 63/244,394, filed on Sep. 15, 2021.
(60) Provisional application No. 63/281,987, filed on Nov. 22, 2021.

(51) Int. Cl. G06V 20/40; A61B 34/00; A61B 90/00; G06V 10/75; G06V 10/762; G06V 10/82

(52) U.S. Cl. CPC G06V 20/46; A61B 34/25; A61B 90/361; G06V 10/75; G06V 10/7625; G06V 10/82; G06V 20/41; A61B 2034/256; A61B 2090/364; A61B 2090/373; G06V 2201/03

(57) ABSTRACT

Methods and apparatuses (e.g., devices and systems, including software) for automatically detecting a one or more features from a video (video file, video stream, etc.) of a surgical procedure. In some examples these methods and apparatuses may include identifying a stage of a surgical procedure (e.g., a surgical stage) of a video or portion of a video of a surgical procedure.

### CLAIM OF PRIORITY

This patent application claims priority to U.S. Provisional Patent Application No. 63/244,385, titled “SYSTEM AND METHOD FOR DETECTING A SURGICAL STAGE,” filed on Sep. 15, 2021, U.S. Provisional Patent Application No. 63/244,394, titled “SYSTEM AND METHOD FOR DETECTING IN-BODY PRESENCE IN A SURGICAL PROCEDURE,” filed on Sep. 15, 2021, and U.S. Provisional Patent Application No. 63/281,987, titled “SYSTEM AND METHOD FOR SEARCHING AND PRESENTING SURGICAL IMAGES,” filed on Nov. 22, 2021, each of which is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

It is becoming increasingly common and useful to record and view, both in real time and later, medical procedures. For example, devices such as endoscopes and surgical microscopes for observing the surgical site during medical procedures such as surgery have become widespread. General medical observation devices, including but not limited to endoscopes may be used when performing or preparing to perform a medical procedure. The resulting videos may be useful for enhancing the surgical procedure and for both pre-operative and post-operative analysis. However, it may be time consuming to view and analyze such videos.

In recent years, Artificial Intelligence has begun to be developed to be used to process images to recognize features of a human face as well as different anatomical structures in a human body. These AI tools can be used to automatically recognize an anatomical feature to assist an operator during a medical procedure, including in particular in interpreting videos of medical procedures. Computational methods such as machine learning and deep learning algorithms can be used to gather and process information generated in a medical procedure. The hope is to use these videos, and machine-learning agents (e.g., AI algorithms) to help understand, interpret and simplify videos of medical procedures. Current systems and methods are still less than ideal in many respects and may be highly time and computing-resource intensive. What is needed are methods and apparatuses, e.g., systems, that may address these problems.

### SUMMARY OF THE DISCLOSURE

Described herein are methods and apparatuses (e.g., devices and systems, including software) related generally to the field of surgery and more specifically to automatically detecting a one or more features from a video (video file, video stream, etc.) of a surgical procedure. In some examples these methods and apparatuses may include identifying a stage of a surgical procedure (e.g., a surgical stage) of a video or portion of a video of a surgical procedure. Also described herein are methods for automatically detecting in-body presence in a surgical procedure in the field of surgery.

For example, described herein are method of automatically identifying a feature from a video of a surgical procedure, the method comprising: receiving, by a processor, a reference to be searched; identifying one or more descriptors from the reference; searching for a correlation between the one or more descriptors from the reference and clusters of one or more descriptors from a bulk video frame set, wherein the bulk video frame set comprises a plurality of sampled video frames from the video of the surgical procedure, wherein the clusters of one or more descriptors from the bulk video frame set have been clustered by the one or more descriptors from the bulk video frame set; selecting one or more images from the bulk video frame set based on the correlation; and outputting the one or more images.

In any of these method or apparatuses configured to perform them (e.g., systems), receiving the reference may comprise receiving a reference image. The reference image may be of one or more of: an MRI scan image, an x-ray image, a video frame, a photograph, or a combination of any of these.

For example, a method of automatically identifying a feature from a video of a surgical procedure may include: receiving, by a processor, a reference image to be searched; identifying one or more descriptors from the reference image; searching for a correlation between the one or more descriptors from the reference image and clusters of one or more descriptors from a bulk video frame set, wherein the bulk video frame set comprises a plurality of sampled video frames from the video of the surgical procedure that have each been translated into the one or more descriptors and clustered by the one or more descriptors from the bulk video frame set, further wherein the plurality of sampled video frames have been paired with a set of metadata; selecting one or more images from the bulk video frame set based on the correlation; and outputting the one or more images and their corresponding metadata for display.

In any of these methods or apparatuses configured to perform them, searching for the correlation may comprise searching using a machine-learning agent. The machine-learning agent may be used for identifying the one or more descriptors, and the same or a different machine learning agent may be used for searching for the correlation.

Forming the bulk video frame set by sampling the video frames from the video of the surgical procedure. Sampling may be done at any appropriate rate, constant or adjustable. For example, the plurality of sampled video frames from the video of the surgical procedure forming the bulk video frame set may have been sampled at a frame rate of between 1 and 10 frames per second.

Any of these methods or apparatuses configured to perform them may include clustering the one or more descriptors from the bulk video frame set. Clustering may be performed by a machine-learning agent.

In any of these methods or apparatuses configured to perform them, outputting the one or more images may further comprise modifying the video of a surgical procedure to indicate the reference. For example, the video may be modified to label and/or flag the reference (e.g., reference image or portion of the reference image). Modifying the video may include adding the metadata or marking the video with the metadata. In any of these methods and apparatuses, outputting may further comprise displaying the one or more images.

As described herein, the clusters of one or more descriptors may be hierarchical. In any of these method and apparatuses searching for the correlation may comprise performing semantic searching. Identifying one or more descriptors from the reference may comprise identifying fc7 descriptors; e.g., using inputs to a last layer of a neural network applied to the reference to identify fc7 descriptors.

The bulk video frame set may comprise sampled video frames from a portion of the video of the surgical procedure. The searching for the correlation may comprise performing a semantic search.

Any of these methods or apparatuses configured to perform them may include identifying a surgical stage from the video of the surgical procedure.

In general, also described herein are apparatuses (e.g., systems) configured to perform any of these methods. For example, a system as described herein may include: one or more processors; a memory coupled to the one or more processors, the memory storing computer-program instructions, that, when executed by the one or more processors, perform a computer-implemented method of automatically identifying a feature from a video of a surgical procedure comprising: receiving, by a processor, a reference to be searched; identifying one or more descriptors from the reference; searching for a correlation between the one or more descriptors from the reference and clusters of one or more descriptors from a bulk video frame set, wherein the bulk video frame set comprises a plurality of sampled video frames from the video of the surgical procedure, wherein the clusters of one or more descriptors from the bulk video frame set have been clustered by the one or more descriptors from the bulk video frame set; selecting one or more images from the bulk video frame set based on the correlation; and outputting the one or more images.

These systems, including the computer-implemented methods stored thereon, may include instructions for performing any of the methods described herein. For example, any of these systems may be configured to receive a reference image (e.g., an image of one or more of: an MRI scan image, an x-ray image, a video frame, a photograph, or a combination of any of these).

In any of these systems, searching for the correlation may comprise searching using a machine-learning agent. The system may be configured to perform the computer-implemented method further comprising forming the bulk video frame set by sampling the video frames from the video of the surgical procedure. The plurality of sampled video frames from the video of the surgical procedure forming the bulk video frame set may have been sampled at a frame rate of between 1 and 10 frames per second.

The computer-implemented method may further be configured to cluster the one or more descriptors from the bulk video frame set. In any of these systems, outputting the one or more images may further comprise modifying the video of a surgical procedure to indicate the reference. In some examples outputting further comprises displaying the one or more images.

As mentioned above, the clusters of one or more descriptors may be hierarchical. The system may be configured to identify one or more descriptors from the reference comprises using inputs to a last layer of a neural network applied to the reference to identify fc7 descriptors. The system may be further configured to search for the correlation by performing semantic searching. In any of these systems, the bulk video frame set may comprise sampled video frames from a portion of the video of the surgical procedure. The searching for the correlation may comprise performing a semantic search. The computer-implemented method performed by the system may further comprise identifying a surgical stage from the video of the surgical procedure.

For example, a system may include: one or more processors; a memory coupled to the one or more processors, the memory storing computer-program instructions, that, when executed by the one or more processors, perform a computer-implemented method of automatically identifying a feature from a video of a surgical procedure comprising: receiving, by a processor, a reference image to be searched; identifying one or more descriptors from the reference image; searching for a correlation between the one or more descriptors from the reference image and clusters of one or more descriptors from a bulk video frame set, wherein the bulk video frame set comprises a plurality of sampled video frames from the video of the surgical procedure that have each been translated into the one or more descriptors and clustered by the one or more descriptors from the bulk video frame set, further wherein the plurality of sampled video frames have been paired with a set of metadata; selecting one or more images from the bulk video frame set based on the correlation; and outputting the one or more images and their corresponding metadata for display.

A non-transitory computer-readable medium including contents that are configured to cause one or more processors to perform a method of automatically identifying a feature from a video of a surgical procedure comprising: receiving, by a processor, a reference to be searched; identifying one or more descriptors from the reference; searching for a correlation between the one or more descriptors from the reference and clusters of one or more descriptors from a bulk video frame set, wherein the bulk video frame set comprises a plurality of sampled video frames from the video of the surgical procedure, wherein the clusters of one or more descriptors from the bulk video frame set have been clustered by the one or more descriptors from the bulk video frame set; selecting one or more images from the bulk video frame set based on the correlation; and outputting the one or more images.

Also described herein are non-transitory computer-readable medium including contents that are configured to cause one or more processors to perform any of these methods.

Also described herein are method and apparatuses (e.g., devices and systems, including software, hardware and firmware) for identifying a surgical stage from a video. For example a method of identifying a surgical stage from a video (which may equivalently be referred to as a method of marking or labeling a surgical stage of a video of s surgical procedure) may include: clustering the video to form one or more clusters; associating one or more semantic tags with the one or more clusters using a machine-language agent trained on video images of medical procedures arranged into clusters that have associated semantic tags; identifying one or more surgical stages from the one or more clusters using the semantic tags associated with each of the one or more clusters; and outputting the one or more surgical stages corresponding to the video. Outputting may further comprise modifying the video to indicate the one or more surgical stages. In some examples outputting further comprises displaying the one or more surgical stages.

The one or more clusters may be hierarchical, and the semantic tags form an ontology. Clustering the video to form one or more clusters may comprise feeding frames of the video into a neural network and using inputs to a last layer of the neural network to generate one or more descriptors that are used to cluster the video.

Identifying the one or more surgical stages may comprise performing semantic searching. In any of these methods the video may comprise a portion of a longer surgical procedure video. Clustering the video to form one or more clusters and associating one or more semantic tags with the one or more clusters may be performed using an online, remote processor.

For example, a system for identifying a surgical stage from a video may include: one or more processors; a memory coupled to the one or more processors, the memory storing computer-program instructions, that, when executed by the one or more processors, perform a computer-implemented method comprising: clustering a video of a medical surgery to form one or more clusters; associating one or more semantic tags with the one or more clusters using a machine-language agent trained on video images of medical procedures arranged into clusters that have associated semantic tags; identifying one or more surgical stages from the one or more clusters using the semantic tags associated with each of the one or more clusters; and outputting the one or more surgical stages corresponding to the video.

Also described herein are non-transitory computer-readable medium including contents that are configured to cause one or more processors to perform any of the methods for identifying a surgical stage from a video, for example: clustering a video of a medical surgery to form one or more clusters; associating one or more semantic tags with the one or more clusters using a machine-language agent trained on video images of medical procedures arranged into clusters that have associated semantic tags; identifying one or more surgical stages from the one or more clusters using the semantic tags associated with each of the one or more clusters; and outputting the one or more surgical stages corresponding to the video.

All of the methods and apparatuses described herein, in any combination, are herein contemplated and can be used to achieve the benefits as described herein.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIG. 1 is a schematic example of a method of automatically identifying a feature from a video of a surgical procedure.

FIG. 2 is a schematic representation of an example system architecture and operating environment for automatically identifying a feature from a video of a surgical procedure.

FIG. 3 schematically illustrates another example of an apparatus for automatically identifying a feature from a video of a surgical procedure as described herein.

FIGS. 4A-4D schematically illustrate examples of a portion of hierarchical clustering algorithm that may be used for identifying a surgical stage from a video.

FIGS. 5A-5C schematically illustrate examples of a portion of a hierarchical clustering algorithm that may be used for identifying a surgical stage from a video.

FIG. 6 schematically illustrates an example of a method as described herein for identifying a surgical stage from a video.

FIG. 7 schematically illustrates one example of a method for automatically detecting in-body presence in a surgical procedure in the field of surgery.

### DETAILED DESCRIPTION

Described herein are methods and apparatuses for efficiently and effectively searching and presenting images from surgical procedures. These methods may be particularly advantageous as compared to other techniques and may be configured specifically for use with medical procedures in a manner that reduces the time to identify particular images, as well as the accuracy of identifying matches. These methods may be used for any type of surgical procedure, including minimally invasive, open and non-invasive surgical procedures. Non-limiting examples of such surgeries may include: bariatric surgery, breast surgery, colon & rectal surgery, endocrine surgery, general surgery, gynecological surgery, hand surgery, head & neck surgery, hernia surgery, neurosurgery, orthopedic surgery, ophthalmological surgery, outpatient surgery, pediatric surgery, plastic & reconstructive surgery, robotic surgery, thoracic surgery, trauma surgery, urologic surgery, vascular surgery, etc.

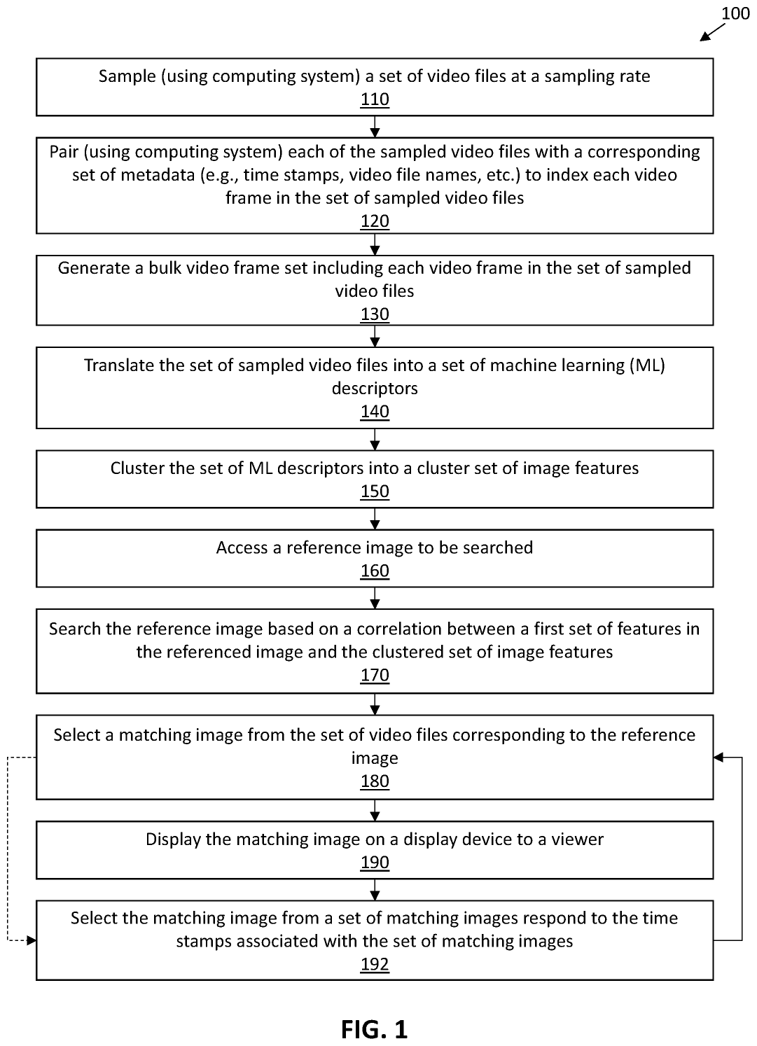

In general, described herein are method of automatically identifying a feature from a video of a surgical procedure. For example, FIG. 1 illustrates one example of a method 100 for searching and presenting surgical images as described herein. Any of the steps of the methods described herein may be performed by a computer system that is configured to perform these steps. For example, these methods may include sampling a set of video files (and/or video streams) at a sampling rate 110. The sampling rate may be predetermined, or adjustable (e.g., user adjustable). The method may further include pairing each of the set of sampled video files (or video streams) with a corresponding set of metadata, including, but not limited to, timestamps and/or video file names, to index each video frame in the set of sampled video files 120. Any of these methods may then include generating a bulk video frame set including each video frame in the set of sampled video files (or video stream(s)) 130. The set of sampled video files and/or video stream(s) may be translated (e.g., by the computer system) into a set of machine-learning (ML) descriptors 140. The ML descriptors may be clustered into a clustered set of image features 150.

In any of these methods, a reference image may be searched 160. The reference image may be searched based on a correlation between a first set of features in the referenced image and the clustered set of image features 170. Thus, any of these methods may include searching the reference image based upon a correlation between a first set of features in the reference image and the clustered set of image features. The method may further include selecting a matching image from the set of video files (or video stream) that corresponds to the reference image in 180 and displaying the matching image on a display device to a viewer in 190. In some examples these methods may also include selecting the matching image from a set of matching images in response to the time stamps associated with the set of matching images 192.

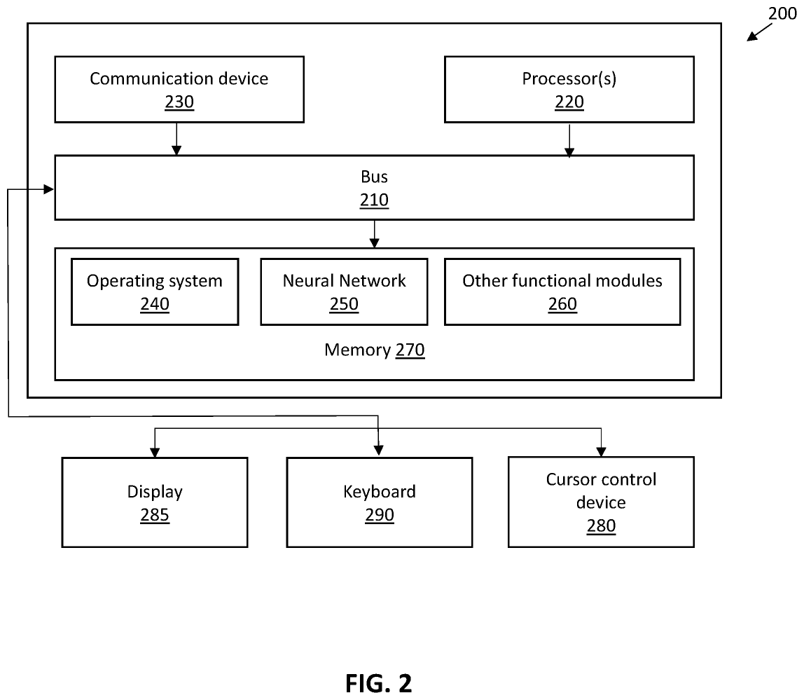

Generally, a computer system 200 (see, e.g., FIG. 2) can perform the methods described herein in order to efficiently and accurately conduct image-based searches through video segments of surgical events and may present the results of the image-based searches. For example, these results may be part of a pre- or post-operative consultation which may include various users and/or viewer users (e.g., surgeons, surgical staff, patients). A computer system 200 as described herein represents a significant technological advances in the field of medical imaging as it may quickly and accurately provide correlations that were previously not possible or were not possible within a reasonable amount of time or using a reasonable amount of processing resources. In general, these methods and apparatuses (e.g., systems) may use one or more machine learning agents to use machine learning techniques (e.g., deep neural network architectures) to conduct image searches in large video-based data sets.

In contrast, traditional computer systems may utilize machine learning techniques to execute machine vision applications, in which the accompanying algorithm is trained (or untrained) to identify objects or features within an image such that the computer system can autonomously make decisions based upon the image input. That is, traditional machine vision techniques are employed for object or feature identification or recognition.

Traditional image-based searching requires substantial computing resources even for still images. In applications in which the searchable field is large, unstructured, homogenous, and expert-based (e.g., surgical videos), the task of autonomously searching for an image requires even more computing power, bandwidth, and time. Traditional image-based searching can therefore overwhelm most computer systems.

By way of comparison, the computer system 200 described herein solves these technical challenges by conducting image-based searches at an abstract level defined within an image processing algorithm. Rather than attempt to search through video files to find a match for a reference image, the computer system 200 executes blocks (as describe in FIG. 1) to decompose, segment, and/or sample the video file, transform images from the video file into a set of abstract descriptors, transform a reference image into a reference abstract descriptor, and then compare the reference abstract descriptor to the set of abstract descriptors to find a best match. In any of these examples, the computer system 200 can solve the foregoing technical challenges by filtering subsets of the set of abstract descriptors within the time domain, as sequential frames within a video file are likely to contain similar or substantially similar information. This may enhance the speed and efficiency.

The computer system 200 can be used in pre- and post-operative consultations with any number of interested parties. Rather than scan through entire surgical video files (or video steam) however, the computer system 200 can execute the method described herein to find the relevant portions of the video file and direct the user to those portions accurately and efficiently.

For example, a surgeon may wish to conduct a pre-surgical interview with a patient in which the surgeon can utilize prior surgery videos and a patient-specific reference image to illustrate to the patient how certain aspects of the surgery are expected to unfold.

Conversely, a surgeon may wish to conduct a post-surgical review with the patient in while the surgeon can utilize video of the patient's surgery and a patient-specific reference image to illustrate to the patient how the surgery actually transpired.

In another example, a surgeon, surgical staff, surgical instructor, or practice manager may wish to conduct a post-operative study, or a series of post-operative studies based upon sets of videos of surgical procedures to ensure best surgical practices are followed and/or the surgical staff is practicing within prescribed risk guidelines. For example, a study lead can operate the computer system 200 described herein to search within sets of video files for relevant portions thereof based upon input reference images or sets of reference images.

In yet another example, a hospital system or insurance administrator can operate the computer system 200 (or sets of computer systems) to ensure surgical best practices are being followed and/or policies and procedures are being followed. For example, a hospital administrator can operate the computer system 200 described herein to search within sets of video files for relevant portions thereof based upon input reference images or sets of reference images.

### Video File Sampling

As discussed above in reference to FIG. 1, in an example implementation, the methods described herein can include sampling a set of video files at a sampling rate 110. The set of video files can include digital video files of surgical procedures, such as endoscopic, arthroscopic, or other image-based or image-guided surgeries and may include video streams (live or recorded). In one example implementation, the computer system 200 can sample the set of video files at a fixed sampling rate, such as 2 frames per second. However, the computer system 200 can alternatively adjust, modify, or alter the fixed sampling rate to a variable sampling rate or a different fixed sampling rate (e.g., 3 frames per second), depending upon a user (surgeon or surgical staff) request, the type of surgery being imaged, and/or the time domain of interest in the search methodologies described herein.

The methods described herein can also include pairing each of the set of sampled video files with a corresponding set of metadata, including timestamps and video file names, to index each video frame in the set of sampled video files 120. Accordingly, the computer system 200 can, for each video frame sampled, generate, create, and/or index the video frame according to at least a video title and a time stamp such that each video frame is associated with a sequence of neighboring (e.g., in the temporal domain) video frames.

The methods described herein may also include generating a bulk video frame set including each video frame in the set of sampled video files 130. In one example implementation, the computer system 200 can concatenate the entire set of video frames derived from the set of sampled video files in order to generate a bulk video frame set. Although the computer system 200 may mix and compile the entire set of video frames into the bulk video frame set, each individual video frame can still be associated with its metadata (e.g., file name, time stamp, etc.). Accordingly, the computer system 200 can concurrently store the metadata for each video frame in a separate data structure (e.g., a dictionary data structure) including a unique video frame index.

As described in detail below, in the examples described below, the computer system 200 can perform these methods on or within the bulk video frame set. Alternatively, the computer system 200 can perform these methods on segments, portions, or subsets of the bulk video frame set. For example these methods may be performed on patches or sub-regions of interest.

### Video File Translation

The methods and apparatuses described herein can further include translating the set of sampled video files into a set of machine-learning (ML) descriptors 140. For example, the computer system 200 can transform, represent, or convert each image in the set of sampled video files into an abstracted descriptor that can be readily searched in place of the raw image data. The one or more abstracted descriptors may be linked to each image (“frame”). As described in further detail below, the computer system 200 can be configured to output and store an abstracted descriptor of each image (e.g., a representation of the image data at an fc7 stage of a neural network, e.g., the next-to last layer/stage), which in turn can be measured against a similarly abstracted rendition of a reference image. Therefore, in conducting an image search according to the example implementation of the methods described herein, the computer system can implement and/or execute techniques described herein to compare and/or match abstracted descriptors of the respective images rather than compare and/or match the visual features of the images.

### Image Preparation

In one alternative variation of the example implementation, the computer system 200 translates the set of sampled video files into a set of machine learning (ML) descriptors by standardizing the data in the bulk video frame set. Subsets of images within the bulk video frame are sampled from video files of different surgical procedures conducted with different types of cameras and captured and/or rendered at different resolutions, aspect ratios, brightness, color, etc. Accordingly, the computer system 100 can translate the set of sampled video files into a set of machine learning (ML) descriptors by normalizing or standardizing each frame in the bulk video frame set, for example by cropping, centering, adjusting color, and/or adjusting contrast for each frame. In another alternative variation of the example implementation, the computer system 200 can normalize or standardize the bulk video frame set according to a set of industry-standard parameters, such as those described in the PyTorch software application. Alternatively, the computer system 200 can normalize or standardize the bulk video frame set according to customized or surgery-dependent parameters.

### Neural Network Training

In another alternative variation of the example implementation, the computer system 200 can translate the set of sampled video files into a set of machine learning (ML) descriptors by training a neural network to receive and classify the images within the bulk video frame set. For example, the computer system can receive or access a pre-trained deep neural network configured for layered image analysis. In yet another alternative variation of the example implementation, the computer system can receive or access a pretrained AlexNet deep-neural network that was trained on an ImageNet database. In this alternative variation of the example implementation, the computer system can access the pretrained AlexNet deep neural network directly from an associated PyTorch library.

In another alternative variation of the example implementation, the computer system can translate the set of sampled video files into a set of machine learning (ML) descriptors by tuning the pre-trained neural network with a prior set of surgical images. For example, the computer system can access a prior set of endoscopic, arthroscopic, or other surgical images from a database. Alternatively, the computer system can access a prior set of video files, sample the video files as described above, and normalize the resulting video frame data as described above. For example, the computer system can: access and/or generate a set of labeled endoscopic surgery datasets for the shoulder and knee regions, load the set of labeled images into the pre-trained neural network, and further train and/or tune the pre-trained neural network on the set of labeled images as described below.

In another alternative variation of the example implementation, the computer system can translate the set of sampled video files into a set of machine learning (ML) descriptors by adjusting, maintaining, and/or differentiating a set of weights and/or biases within the pre-trained neural network in order to finely tune the pre-trained neural network to surgical imagery. As noted above, the computer system can access and/or execute a deep neural network, which is defined by a set of layers including a subset of fully connected layers and a subset of non-fully connected layers. Accordingly, to tune the pre-trained neural network the computer system can differentially adjust or maintain the weights and/or biases within the subsets of layers. In yet another alternative variation of the example implementation, the computer system can freeze or fix the non-fully connected layers of the pre-trained neural network such that the weights are fixed during the tuning (re-training) process. In doing so, only the weights within the fully connected layers are updated using the tuning data sets (e.g., surgery-specific images).

In yet another variation of the example implementation, the computer system can translate the set of sampled video files into a set of machine learning (ML) descriptors by ingesting or accessing initial renditions of the set of labeled images and then rotating or transforming the set of labeled images into a second set of rotated renditions of the set of labeled images. The computer system can therefore translate the set of sampled video files into a set of machine learning (ML) descriptors using initial and rotated versions of the same labeled images when tuning the pre-trained neural network. In doing so, the computer system can tune the pre-trained neural network to operate in a rotation-invariant manner when interpreting the reference image. As such, the computer system can tune the deep neural network to operate and/or interpret rotation-invariant image data from surgical images.

### Abstracted Descriptor Generation

In yet another variation of the example implementation, the computer system can translate the set of sampled video files into a set of machine learning (ML) descriptors by generating an abstracted descriptor corresponding to the generalizations of the image data derived in the deepest layer in the deep neural network. For example, after tuning the pre-trained neural network, the computer system can generate a duplicate neural network model substantially identical to the original neural network except for the last layer. In particular, the computer system can generate a new model in which the final fully connected layer, the fc7 layer, constitutes the output of the model. Therefore, rather than a qualitative termination of the neural network (e.g., a classification of the image), the computer system generates an abstract model that forms the basis of a search function as described in detail below.

In operation, the computer system can be configured to disable node dropout and freeze all other parameters such that computer system can utilize the new and final iteration of the abstract model to evaluate the frames by outputting an abstract array corresponding to the fc7 layer weights. For example, the computer system can output a 4096-dimensional array corresponding to the fc7 layer weights when a frame is propagated through the duplicate neural network. Generally, each 4096-dimensional fc7 feature contains generalizable information about the input frame since the feature corresponds to the deepest layer in the duplicate neural network's parameters. As described below, the computer system can then conduct, implement, and direct image-based searching within video data with machine-learning descriptors (at the most generalizable level of the neural network) rather than the pixelated or specific feature layer as is generally practiced.

### Machine-Learning Descriptor Clustering

As discussed above (e.g., and shown in FIG. 1), the example implementation of the methods described herein can further include: clustering the set of machine-learning descriptors into a clustered set of image features in 150. For example, the computer system can feed the fc7 features for each sampled frame into a clustering algorithm to group, cluster, or arrange similar features between frames and thus group, cluster, or arrange similar frames with one another. In one alternative variation of the example implementation, the computer system can execute an agglomerative clustering, which does not require the user to specify the number of clusters. Rather, in agglomerative clustering, which is a hierarchical technique, the user can be prompted to specify the depth of the neural network as a parameter, which determines the extent to which the hierarchical relational tree is truncated to yield bins of similar frames.

For example, if a user selects a low depth value, then the computer system will arrange or construct a very complex hierarchy with a relatively large number of sub-branches, meaning that the clustering algorithm will generate relatively more bins/clusters and fewer frames per cluster.

Conversely, if a user selects a high depth value, then the computer system will arrange or construct a less complex hierarchy with a relatively low number of sub-branches, meaning that the clustering algorithm will generate relatively fewer bins/clusters and relatively more frames per cluster.

Alternatively, the computer system can execute other types of clustering algorithms including top-down agglomerative clustering techniques or combinations of agglomerative clustering algorithms with K-means or mean shift clustering techniques.

In another variation of the example implementation, the computer system can cluster the set of ML descriptors into a cluster set of image features by incorporating temporal information and/or designations into the image clustering. For example, as the surgeon proceeds through a surgery, the visual scene at the surgical site will change with time (e.g., tissues are repaired, anchored, sutured, etc.). Accordingly, in this variation of the example implementation, the computer system can associate or attach clinically pertinent metadata to the set of frames within each cluster, including for example a surgical phase (e.g., diagnostic phase, treatment phase, post-treatment examination, etc.) as well as additional contextual data.

### Reference Image Searching

The example methods described herein can also include accessing a reference image to be searched 160. Generally, the reference image can be delivered, transmitted, uploaded, or selected by the computer system in response to a user request or user input (e.g., at the request or input of a surgeon or surgical staff). For example, the reference image can include an MRI scan image, an x-ray image, a video frame, a photograph, or a composite or fusion of any of the foregoing of a patient's anatomy (e.g., a knee or shoulder). The computer system can accept user input and then access the reference image in its original format from a local or remote database, or directly from an imaging device.

The computer system can transform the reference image into a set of features or descriptors at the fc7 level of abstraction. For example, the computer system can normalize the reference image by size and centering and generate an abstracted reference image descriptor corresponding to the generalizations of the reference image data derived in the deepest layer in the deep neural network. As noted above, after tuning the pre-trained neural network, the computer system can generate a duplicate neural network model substantially identical to the original neural network except for the last layer. Therefore, the computer system can readily generate the fc7 data of the abstracted reference image descriptor.

As discussed above, any of these methods can further include searching the reference image based upon a correlation between a first set of features in the reference image and the clustered set of image features 170. In operation, the computer system can receive a prompt from a user to select a depth parameter, which is one of the parameters through which the user can direct the computer system to control the strictness or looseness of the image search. Alternatively, the computer system can recommend a depth parameter based upon prior iterations of the method. In response to a depth selection by the user, the computer system can implement techniques and methods described above to cluster the fc7 features of the reference image and the bulk video frame set and reconstruct the master dictionary to match the structure of the clusters.

In one variation of the example implementation, the computer system can separate the reference image from the bulk video frame clustering since agglomerative clustering is hierarchical and cannot be updated without recalculating the entire hierarchy.

In another variation of the example implementation of the method, once the bulk video frame images have been grouped together into a set of clusters, the computer system can layer a simple centroid-based classifier to find which bin/cluster the reference image's fc7 feature belongs to as the reference image is not a part of the bulk video frame clustering.

In another variation of the example implementation of the method, the computer system can compute, for each cluster, the centroids of the fc7 feature clusters. Generally, the centroids do not necessarily correspond to a real frames' fc7 features since they represent the center of mass of the distribution in 4096-dimensional space. Accordingly, the computer system can select from the set of clusters a representative frame that includes an fc7 feature that minimizes the Euclidean distance to its respective centroid. The computer system can then relate the representative frames' fc7 features to the reference image fc7 feature. Furthermore, the computer system can calculate which representative frame's fc7 feature has the lowest Euclidean distance to the reference image fc7 feature. In response to calculating the lowest Euclidean distance between the representative frame's fc7 feature and the reference image fc7 feature, the computer system can select the cluster that includes the representative frame as the matching cluster, and therefore associated with the matching image within the original set of video frames.

In another variation of the example implementation, the computer system can execute implement and/or execute a trained artificial neural network (ANN) that is configured to automatically segment specific anatomical features of interest while removing and/or ignoring additional or excess anatomical features (e.g., healthy tissues) and/or surgical tools. Through experience and training, a surgeon instinctively ignores excess anatomical tissue(s) and/or tools during procedures while focusing on the surgical site and pathological tissues. Accordingly, the computer system can similarly refine and/or segment images as described above to classify images according to relevant anatomical features to the exclusion of visually dominant but irrelevant objects such as surgical tools in the field of view.

As described above, the example implementation of the method can also include selecting a matching image from the set of video files that corresponds to the reference image and displaying the matching image on a display device to a viewer (e.g., a surgeon, surgical staff, and/or patient).

In one variation of the example implementation, the method can also include selecting the matching image from a set of matching images in response to the time stamps associated with the set of matching images. For example, in searching the reference image based on a correlation between a first set of features in the referenced image and the clustered set of image features and selecting a matching image from the set of video files corresponding to the reference, the computer system can order, rank, or organize a set of frames based upon their respective Euclidean distance to the reference image fc7 feature. Accordingly, in selecting a matching image from the set of video files corresponding to the reference, the computer system can rank the closest match (e.g., lowest Euclidean distance measurement) as the associated image that is presented to the viewer.

However, due to the spatial and temporal characteristics of surgical imagery and the gross similarity of anatomic features, a set of frames can include redundant or extremely similar images and therefore potentially redundant reference features. Accordingly, the computer system 100 can further filter the set of redundant images in response to the time stamps within the metadata associated with each video frame. For example, the computer system can define a temporal threshold about a video frame associated with the closest Euclidean match, compile all frames within the temporal threshold, remove temporally adjacent frames from the output (e.g., frames including timestamps within the threshold), and preserve, render, and/or display the first visited (based upon timestamp information) and thus closest matching frame in the threshold interval.

As noted above, the computer system can also associate frames with temporal and/or clinically pertinent metadata. For example, as a surgeon operates on a pathology, the appearance of that pathology changes from its original state to its repaired state, along with intermediate surgical states. The computer system can associate the reference image with a user-selected metadata phase (e.g., diagnostic phase, treatment phase, post-treatment examination). As such, when searching for an image with a pathology (e.g. diagnostic phase), the computer system can prioritize images within image clusters associated with the diagnostic phase metadata. Alternatively, when searching for an image with a specific surgical technique or approach (e.g., treatment phase), the computer system can prioritize image within image clusters associated with the treatment phase metadata.

### Operating Environment and Architecture

As shown in FIG. 2, the computer system 200 can execute the methods described herein within an exemplary operating environment or architecture. As shown, the computer system 200 can include any one or more of the computing systems depicted and/or described herein. An example computer system 200 may include a bus 210 or other communication mechanism for communicating information, and processor(s) 220 coupled to bus 210 for processing information. Exemplary processor(s) 220 can be any type of general or specific purpose processor, including a Central Processing Unit (CPU), an Application Specific Integrated Circuit (ASIC), a Field Programmable Gate Array (FPGA), a Graphics Processing Unit (GPU), multiple instances thereof, and/or any combination thereof. Exemplary processor(s) 220 can also have multiple processing cores, and at least some of the cores can be configured to perform specific functions. Multi-parallel processing can be used in some example implementations. In certain example implementations, at least one of processor(s) 220 can be a neuromorphic circuit that includes processing elements that mimic biological neurons. In some example implementations, neuromorphic circuits do not require the typical components of a Von Neumann computing architecture.

As shown in FIG. 2, an exemplary computer system 200 further includes a memory 270 for storing information and instructions to be executed by processor(s) 220. Memory 270 can include of any combination of Random Access Memory (RAM), Read Only Memory (ROM), flash memory, cache, static storage such as a magnetic or optical disk, or any other types of non-transitory computer-readable media or combinations thereof. Non-transitory computer-readable media can be any available media that can be accessed by processor(s) 220 and can include volatile media, non-volatile media, or both. The media can also be removable, non-removable, or both.

Additionally, the exemplary computer system 200 may include a communication device 230, such as a transceiver, to provide access to a communications network via a wireless and/or wired connection. In some example implementations, the communication device 230 can be configured to use Frequency Division Multiple Access (FDMA), Single Carrier FDMA (SC-FDMA), Time Division Multiple Access (TDMA), Code Division Multiple Access (CDMA), Orthogonal Frequency Division Multiplexing (OFDM), Orthogonal Frequency Division Multiple Access (OFDMA), Global System for Mobile (GSM) communications, General Packet Radio Service (GPRS), Universal Mobile Telecommunications System (UMTS), cdma2000, Wideband CDMA (W-CDMA), High-Speed Downlink Packet Access (HSDPA), High-Speed Uplink Packet Access (HSUPA), High-Speed Packet Access (HSPA), Long Term Evolution (LTE), LTE Advanced (LTE-A), 802.11x, Wi-Fi, Zigbee, Ultra-WideBand (UWB), 802.16x, 802.15, Home Node-B (HnB), Bluetooth, Radio Frequency Identification (RFID), Infrared Data Association (IrDA), Near-Field Communications (NFC), fifth generation (5G), New Radio (NR), any combination thereof, and/or any other currently existing or future-implemented communications standard and/or protocol without deviating from the scope of the invention. In some example implementations, the communication device 230 can include one or more antennas that are singular, arrayed, phased, switched, beamforming, beamsteering, a combination thereof, and or any other antenna configuration without deviating from the scope of the invention.

As shown in FIG. 2, exemplary processor(s) 220 can be further coupled via bus 210 to a display 285, such as a plasma display, a Liquid Crystal Display (LCD), a Light Emitting Diode (LED) display, a Field Emission Display (FED), an Organic Light Emitting Diode (OLED) display, a flexible OLED display, a flexible substrate display, a projection display, a 4K display, a high definition display, a Retina™ display, an In-Plane Switching (IPS) display, or any other suitable display for displaying information to a user. Generally, the display 285 can be configured as a touch (haptic) display, a three dimensional (3D) touch display, a multi-input touch display, a multi-touch display, etc. using resistive, capacitive, surface-acoustic wave (SAW) capacitive, infrared, optical imaging, dispersive signal technology, acoustic pulse recognition, frustrated total internal reflection, etc. Any suitable display device and haptic I/O can be used without deviating from the scope of the invention.

As shown in FIG. 2, a keyboard 290 and a cursor control device 280, such as a computer mouse, a touchpad, etc., may be further coupled to the bus 210 to enable a user to interface with the computer system 200. However, in certain example implementations, a physical keyboard and mouse may not be present, and the user can interact with the device solely through the display 285 and/or a touchpad (not shown). Any type and combination of input devices can be used as a matter of design choice.

In some example implementations, the display 285 can include an augmented reality (AR) or virtual reality (VR) headset configured to communicate with the bus 210 and the computer system 200 through wired and/or wireless communication protocols.

In still other example implementations, no physical input device and/or display 285 is present. For instance, the user can interact with the computer system 200 remotely via another computer system in communication therewith, or the computer system 200 can operate autonomously or semi-autonomously with little or no user input.

As shown in FIG. 2, an exemplary memory 270 can store software modules that provide functionality when executed by processor(s) 220. The modules can include an operating system 240 for the computer system 200; a deep neural network module 250 that may be configured to perform all, or part of the processes described herein or derivatives thereof; and one or more additional functional modules 250 that include additional functionality.

Generally, the computer system 200 can be embodied as a server, an embedded computing system, a personal computer, a console, a personal digital assistant (PDA), a cell phone, a tablet computing device, a quantum computing system, or any other suitable computing device, or combination of devices without deviating from the scope of the invention. Presenting the above-described functions as being performed by a system is not intended to limit the scope of the present invention in any way but is intended to provide one example of the many example implementations of the present invention. Indeed, methods, systems, and apparatuses disclosed herein can be implemented in localized and distributed forms consistent with computing technology, including cloud computing systems and/or edge computing systems.

A computer system can be implemented as an “engine” (e.g., an image search engine), as part of an engine, or through multiple engines. As used herein, an engine includes one or more processors or a portion thereof. A portion of one or more processors can include some portion of hardware less than all of the hardware comprising any given one or more processors, such as a subset of registers, the portion of the processor dedicated to one or more threads of a multi-threaded processor, a time slice during which the processor is wholly or partially dedicated to carrying out part of the engine's functionality, or the like. As such, a first engine and a second engine can have one or more dedicated processors, or a first engine and a second engine can share one or more processors with one another or other engines. Depending upon implementation-specific or other considerations, an engine can be centralized, or its functionality distributed. An engine can include hardware, firmware, or software embodied in a computer-readable medium for execution by the processor. The processor transforms data into new data using implemented data structures and methods, such as is described with reference to the figures herein.

The engines described herein, or the engines through which the systems and devices described herein can be implemented, can be cloud-based engines. As used herein, a cloud-based engine is an engine that can run applications and/or functionalities using a cloud-based computing system. All or portions of the applications and/or functionalities can be distributed across multiple computing devices and need not be restricted to only one computing device. In some embodiments, the cloud-based engines can execute functionalities and/or modules that end users access through a web browser or container application without having the functionalities and/or modules installed locally on the end-users' computing devices.

As used herein, datastores are intended to include repositories having any applicable organization of data, including tables, comma-separated values (CSV) files, traditional databases (e.g., SQL), or other applicable known or convenient organizational formats. Datastores can be implemented, for example, as software embodied in a physical computer-readable medium on a specific-purpose machine, in firmware, in hardware, in a combination thereof, or in an applicable known or convenient device or system. Datastore-associated components, such as database interfaces, can be considered “part of” a datastore, part of some other system component, or a combination thereof, though the physical location and other characteristics of datastore-associated components is not critical for an understanding of the techniques described herein.

Datastores can include data structures. As used herein, a data structure is associated with a particular way of storing and organizing data in a computer so that it can be used efficiently within a given context. Data structures are generally based on the ability of a computer to fetch and store data at any place in its memory, specified by an address, a bit string that can be itself stored in memory and manipulated by the program. Thus, some data structures are based on computing the addresses of data items with arithmetic operations; while other data structures are based on storing addresses of data items within the structure itself. Many data structures use both principles, sometimes combined in non-trivial ways. The implementation of a data structure usually entails writing a set of procedures that create and manipulate instances of that structure. The datastores, described herein, can be cloud-based datastores. A cloud based datastore is a datastore that is compatible with cloud-based computing systems and engines. An automated machine learning engine(s) may implement one or more automated agents configured to be trained using a training dataset.

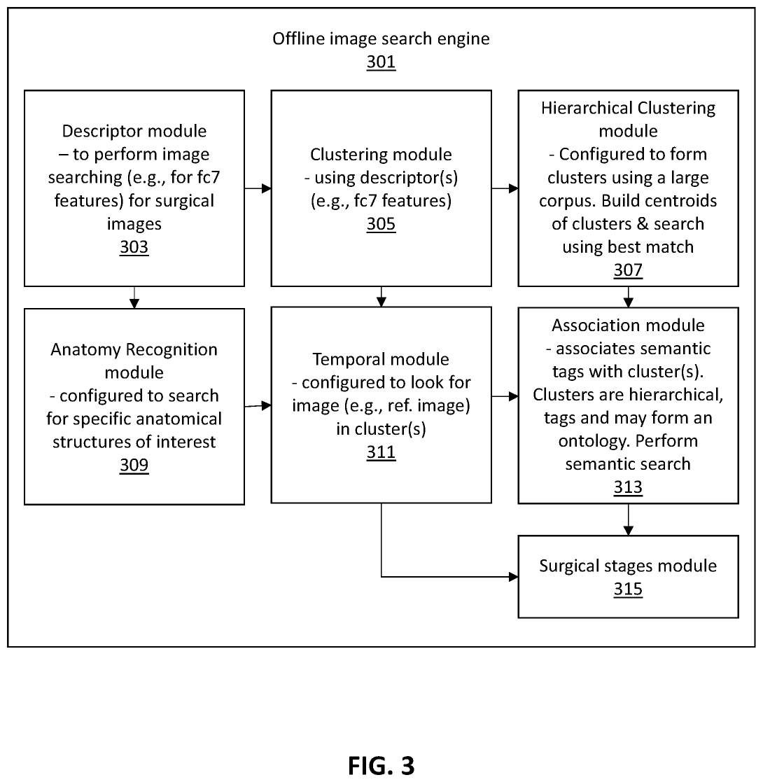

FIG. 3 schematically illustrates another example of an image search (e.g., image search engine) similar to that described above. In this example the image search engine 301 may be referred to as an offline image search engine, as it may be operated offline, e.g., after recording and/or transmitting the image(s). Alternatively, in some examples the image search engine may be an online (or real-time) image search engine. In FIG. 3, the example includes a descriptor module 303 that is configured to perform image searching, e.g., using fc7 features of images. For example, the networks described herein (e.g., ML agents) may be trained on anatomical images having descriptors, such as fc7 descriptors, that are generally tuned for surgical images. The image search engine may receive input of one or more images (e.g., optionally a bulk video frame set, etc. and a reference image to be searched. The input images may be paired with metadata either before being input into the image search engine 301 or the image search engine may include a metadata pairing module to perform this action.

The image search engine may also include an anatomy recognition module configured to search for specific anatomical structures of interest 309 within the input image(s)/bulk images (e.g., video files and/or video streams) after they have been processed by the descriptor module 303.

In any of the apparatuses and systems described herein, the apparatus may include a clustering module 305 that is configured to cluster the set(s) of ML descriptors into one or more cluster set(s) of image features, after the operation of the descriptor module 303. In some examples the clustering module may cluster features using fc7 features, where the fc7 features are used as descriptors.

The image search engine 301 may also include a Temporal module 311 (also referred to as a temporal search engine), that is configured to search the cluster(s) for images corresponding to the reference image (or in some examples, reference video).

A hierarchical clustering module 307 may also be included. The hierarchical clustering module may be configured to form clusters using a large dataset (e.g., large corpus). The hierarchical clustering module 307 may build centroids of clusters and may search using best match.

The image search engine 301 may also include an association module 313. The association module may associate semantic tags with cluster(s). Clusters are hierarchical, tags and may form an ontology. The association image may also perform semantic searching of the clusters using tags.

Finally, the image search engine 301 may also include a surgical stage module 315. The surgical stage module 315 may output the surgical stage based on the search modules (e.g., the temporal search module 311 and/or the association module 313).

The image searching engine may include one or more outputs (which may be processed by an output module, not shown) for outputting the search results for either the temporal and/or schematic searching and/or for outputting the surgical stage. Output may include the metadata/tag data. As described in FIG. 1, the output may include displaying a matching image from a set of video files/video stream, e.g., on a display and/or memory (datastore). The output may be further manipulated, including marked, labeled, etc.

As mentioned above, some of the system features described in this specification have been presented as modules or engines, in order to more particularly emphasize their implementation independence. For example, a module can be implemented as a hardware circuit comprising custom very large scale integration (VLSI) circuits or gate arrays, off-the-shelf semiconductors such as logic chips, transistors, or other discrete components. A module can also be implemented in programmable hardware devices such as field programmable gate arrays, programmable array logic, programmable logic devices, graphics processing units, or the like.

A module can also be at least partially implemented in software for execution by various types of processors. An identified unit of executable code can, for instance, include one or more physical or logical blocks of computer instructions that can, for instance, be organized as an object, procedure, or function. Nevertheless, the executables of an identified module need not be physically located together but can include disparate instructions stored in different locations that, when joined logically together, define the module and achieve the stated purpose for the module. Further, modules can be stored on a computer-readable medium, which can be, for instance, a hard disk drive, flash device, RAM, tape, and/or any other such non-transitory computer-readable medium used to store data without deviating from the scope of the invention.

Generally, a module of executable code could be a single instruction, or many instructions, and can even be distributed over several different code segments, among different programs, and across several memory devices. Similarly, operational data can be identified and illustrated herein within modules and can be embodied in any suitable form and organized within any suitable type of data structure. The operational data can be collected as a single data set or can be distributed over different locations including over different storage devices, and can exist, at least partially, merely as electronic signals on a system or network.

The systems and methods described herein can be embodied and/or implemented at least in part as a machine configured to receive a computer-readable medium storing computer-readable instructions. The instructions can be executed by computer-executable components integrated with the application, applet, host, server, network, website, communication service, communication interface, hardware/firmware/software elements of a user computer or mobile device, wristband, smartphone, or any suitable combination thereof. Other systems and methods of the embodiment can be embodied and/or implemented at least in part as a machine configured to receive a computer-readable medium storing computer-readable instructions. The instructions can be executed by computer-executable components integrated by computer-executable components integrated with apparatuses and networks of the type described above. The computer-readable medium can be stored on any suitable computer readable media such as RAMs, ROMs, flash memory, EEPROMs, optical devices (CD or DVD), hard drives, floppy drives, or any suitable device. The computer-executable component can be a processor, but any suitable dedicated hardware device can (alternatively or additionally) execute the instructions.

In any of these method and apparatuses (e.g., device, systems, etc. including software, hardware and/or firmware), real-time cluster matching and novelty detection may be performed. Generally, the processes and apparatuses for performing them described herein may operate in real- or near-real time. This includes identifying matches to one or more clusters by a reference image or images and/or semantic tag matching with or without a reference image.

Further, the methods and apparatuses described herein may include matching all of the image/video (e.g., all of the filed of view), or a sub-region of the image/video. For example, these methods may be performed using a sub-region or patch. Thus, any of these methods and/or apparatuses may include identifying or limiting to the sub-region or patch. For example, a sub-region or patch of a reference video may be used, or a sub-region or patch of the sampled and/or bulk video may be used. The sub-region or patch may be selected by the user (e.g., manually) or automatically, e.g., to include a relevant region or time.

### Detection of Surgical Stage

Also described herein are methods of detecting (e.g., automatically detecting) one or more surgical stages from a video (or video stream). Generally, detecting the stage or the surgical sub-procedure may be of significant value to hospitals and surgery centers. Signals about the stage of the current surgery in the operating room (OR) may help administrators manage the surgical workflow, e.g., preparing patients waiting to enter surgery, ensuring that the recovery rooms are available, etc.

Any of the apparatuses and methods described herein be configured to detect a surgical stage or may include detecting a surgical stage. For example, a system as described herein may perform the steps of receiving and/or processing a video (e.g., a stream of video frames) from an endoscopic imaging system and applying hierarchical clustering algorithms on the incoming frames to cluster the frames. In some cases the clustering algorithms may be located remotely (e.g., online). The system can execute two or more variations of the online techniques with the stage recognition system.

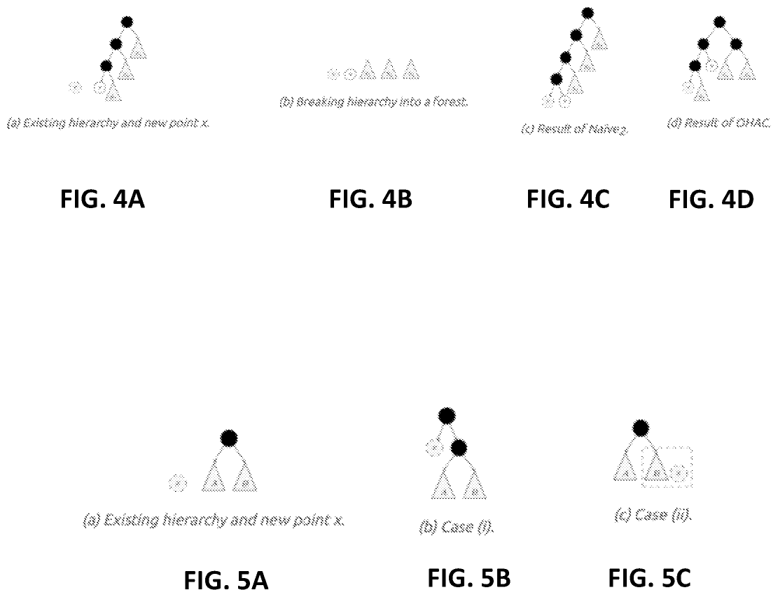

In one variation, the system can execute a top-down algorithm in which the algorithm performs a search from the root towards the leaves of the tree and inserts the image into an existing cluster or creates a leaf/branch if the incoming image is sufficiently distant from existing clusters. An example of these structures are illustrated in FIGS. 4A-4D.

In another example, schematically illustrated in FIGS. 5A-5C, the system can execute (e.g., an online version of) a hierarchical clustering algorithm in which the portions of the hierarchy are merged and rebuilt for each incoming frame of the video feed. In another example implementation, the ‘distance’ between the elements is computed in the multi-dimensional space containing the data. The system can employ a novel distance measure, specifically designed for surgical images. In particular, the system can execute a distance measure that operates on coordinates in a 4096 (e.g., any arbitrarily set) space. The system can also feed each input frame into a neural network. Furthermore, the system can remove the last layer of the network and the inputs to the last layer are captured into a vector. The system can then extract features from the vector, called fc7 features, containing information about the input frame at varying levels of abstraction.

In another variation of an example implementation, the system can execute the distance measure with a deep neural network, e.g., UNet, which has been trained to recognize anatomical structures in arthroscopic procedures. Therefore, the fc7 features are highly specialized and reflect the images in a surgery.

In another variation of an example implementation, the system can create clusters containing images with similar characteristics. By combining temporal information into the clustering techniques as an additional dimension, the system can generate the clusters to reflect sequences of frames which display similar anatomical structures, and which are temporally connected. Furthermore, when clusters from neighboring branches of the hierarchical tree are considered together, they represent slow changes in the surgical field of view.

In another variation, the system can recognize a state of the surgical procedure (live or recorded) by applying a semantic relationship to the clusters. The system can execute the novel distance measure to determine to which surgery stage a newly formed cluster belongs. This cluster, being populated in time, represents a distinct stage in the surgery based on the image and temporal proximity with their neighboring frames.

As the hierarchical cluster is being constructed, the system may test the centroids of the clusters below the non-leaf nodes against a reference catalog of images that contains representative images from various stages in each surgical procedure. Additionally, each of the reference images can also contain a clinically significant tag/label describing the stage of the surgery.

In another variation of the example implementation, in response to the system detecting a matching reference image for a cluster that is being newly formed, the system may output the label corresponding to the reference image as the surgery stage.

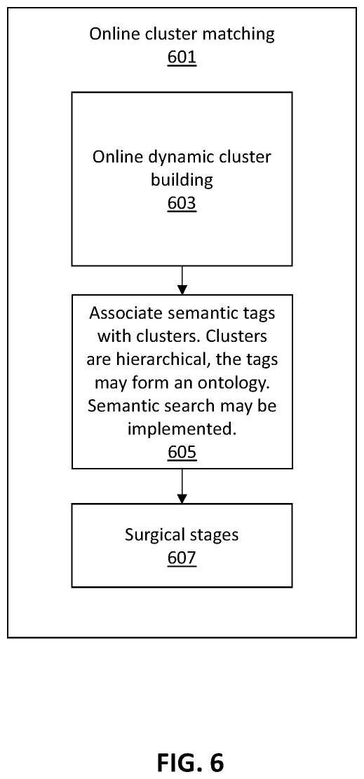

Thus, any of the methods and apparatuses that include stage recognition may use cluster matching to determine one or more surgical stages. For example, FIG. 6 schematically an example of cluster matching. This example may be particularly well suited for online (e.g., remote) cluster matching 601. As shown, the process for stage recognition may include dynamic cluster building 603. In general, the larger the training dataset (e.g., videos) the more stable the clusters may be. Cluster building may be performed using a remote (e.g., online) processor running any of the clustering processes described herein. The method (or a system performing the method) may also include associating semantic tags with the clusters 605. The clusters may be hierarchical, and tags may form an ontology. In general, the any appropriate information content may be used for clustering and as part of the tags. For example, the tags may refer to anatomic information, procedural information, tool (e.g., surgical tool) information, etc.

As a result of the clustering and matching of cluster(s) with the tags (or using tags), one or more surgical stages may be identified 607.

In general, cluster selection may be automatic or semi-automatic (e.g., with user input/confirmation), providing for automatic stage recognition.

### Detecting an In-Body Presence

Also described herein are methods and apparatuses (e.g., systems) for detecting if a camera (in the given frame) is inside or outside the body. Generally, hospitals and large surgery centers may utilize hardware and software components to stream endoscopic surgery videos for analysis and for record keeping purposes. Currently, hospitals and surgery centers rely on the surgeons or the surgeons' assistants to manually start and stop recording the surgeries.

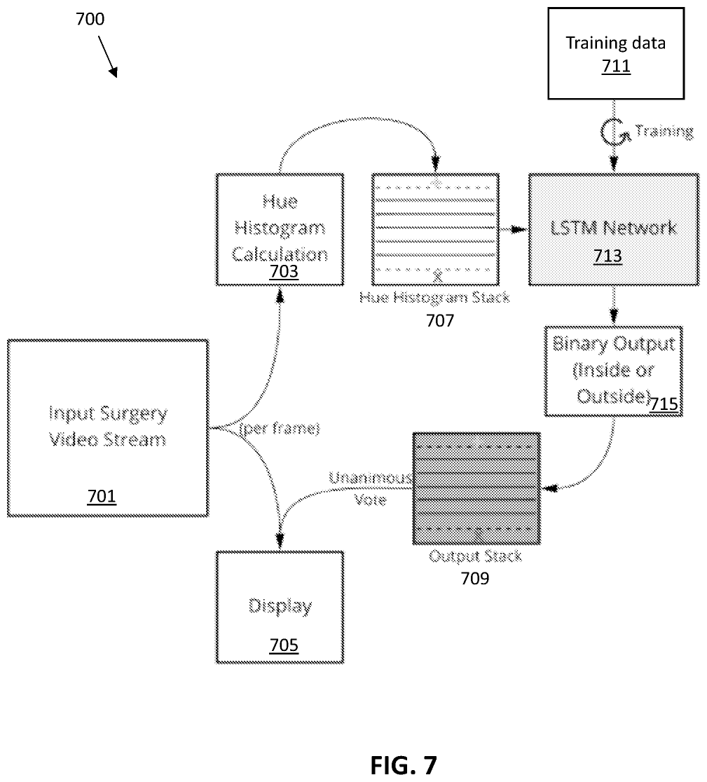

As shown in FIG. 7, an apparatus (e.g., system) can detect in-body presence in a surgical procedure (e.g., during the surgical procedure). The system may do this by processing an input video stream 701 frame-by-frame (or sampling a subset of frames) and running a supervised binary classification algorithm to determine whether the camera in the given frame is inside or outside the body.

In one example implementation, the system 700 includes an algorithm pipeline that first converts the frame to a hue histogram 703, which is a representation of the color distribution in the frame. The system can convert the image to a hue histogram (e.g., hue histogram stack 707) to maintain generality of the descriptor, which may reduce the complexity and computational time. A more specific descriptor, such as the full image, may require a significant amount of training data to teach the supervised classification algorithm. By using a more general descriptor, the system may avoid overfitting, allowing the model to be generalized to different types of surgeries, and reduce the amount of training data needed. Other descriptors (alternatively or additionally to hue, may be used, including intensity, etc.

In some examples, the system accrues the hue histograms per frame into a temporal sliding-window stack, which may be fed into a long short-term memory (LSTM) neural network, which additionally smooths the transitions between inside and outside of the body. Thus by using a sliding window of frames as an input, the system can dilute the new information from an incoming frame so that the binary classification algorithm is robust to instabilities.

In one example implementation, the system executes an LSTM neural network 713 based on the temporally contextual nature of surgery scope removal and insertion into the body. Generally, LSTM networks take sequences as input, and information is passed between timesteps in the sequence. In a surgery, a scope/camera will always traverse the same anatomy when inserting and/or removing the scope, and travel through the trocar in both insertion and removal. Therefore, the system can pass information between timesteps such that it can predict the classification of the anatomy in a highly contextual manner. The system can execute the foregoing methods and techniques such that classification is performed in real-time with the input video stream.

In another example, the system calculates binary classification output per frame 715 at the LSTM network and accumulates the outputs in another sliding window stack 709. In some examples, the system will adjust the real-time classification in response to a unanimous vote of all per-frame outputs in the stack.

In some example implementations, the foregoing technique may be a strict smoothing system that substantially eliminates instabilities which are shorter than the temporal width of the sliding window stack. For example, in a surgery, there are often rapid movements which could cause temporary misclassification. By applying a unanimous voting output stack 709, the system may allow “intent” to be resolved in scope removal/insertion.

In some example implementations, the system can implement training of the LSTM neural network on a set of surgeries (e.g., endoscopic knee-surgery videos and/or mock endoscopic surgery videos) 711.

Any of the systems and methods described herein can be embodied and/or implemented at least in part as a machine configured to receive a computer-readable medium storing computer-readable instructions. The instructions can be executed by computer-executable components integrated with the application, applet, host, server, network, website, communication service, communication interface, hardware/firmware/software elements of a user computer or mobile device, wristband, smartphone, or any suitable combination thereof. Other systems and methods of the embodiment can be embodied and/or implemented at least in part as a machine configured to receive a computer-readable medium storing computer-readable instructions. The instructions can be executed by computer-executable components integrated by computer-executable components integrated with apparatuses and networks of the type described above. The computer-readable medium can be stored on any suitable computer readable media such as RAMs, ROMs, flash memory, EEPROMs, optical devices (CD or DVD), hard drives, floppy drives, or any suitable device. The computer-executable component can be a processor, but any suitable dedicated hardware device can (alternatively or additionally) execute the instructions.

It should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

The process parameters and sequence of steps described and/or illustrated herein are given by way of example only and can be varied as desired. For example, while the steps illustrated and/or described herein may be shown or discussed in a particular order, these steps do not necessarily need to be performed in the order illustrated or discussed. The various example methods described and/or illustrated herein may also omit one or more of the steps described or illustrated herein or include additional steps in addition to those disclosed.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like. For example, any of the methods described herein may be performed, at least in part, by an apparatus including one or more processors having a memory storing a non-transitory computer-readable storage medium storing a set of instructions for the processes(s) of the method.

While various embodiments have been described and/or illustrated herein in the context of fully functional computing systems, one or more of these example embodiments may be distributed as a program product in a variety of forms, regardless of the particular type of computer-readable media used to actually carry out the distribution. The embodiments disclosed herein may also be implemented using software modules that perform certain tasks. These software modules may include script, batch, or other executable files that may be stored on a computer-readable storage medium or in a computing system. In some embodiments, these software modules may configure a computing system to perform one or more of the example embodiments disclosed herein.

As described herein, the computing devices and systems described and/or illustrated herein broadly represent any type or form of computing device or system capable of executing computer-readable instructions, such as those contained within the modules described herein. In their most basic configuration, these computing device(s) may each comprise at least one memory device and at least one physical processor.

The term “memory” or “memory device,” as used herein, generally represents any type or form of volatile or non-volatile storage device or medium capable of storing data and/or computer-readable instructions. In one example, a memory device may store, load, and/or maintain one or more of the modules described herein. Examples of memory devices comprise, without limitation, Random Access Memory (RAM), Read Only Memory (ROM), flash memory, Hard Disk Drives (HDDs), Solid-State Drives (SSDs), optical disk drives, caches, variations or combinations of one or more of the same, or any other suitable storage memory.

In addition, the term “processor” or “physical processor,” as used herein, generally refers to any type or form of hardware-implemented processing unit capable of interpreting and/or executing computer-readable instructions. In one example, a physical processor may access and/or modify one or more modules stored in the above-described memory device. Examples of physical processors comprise, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, or any other suitable physical processor.

Although illustrated as separate elements, the method steps described and/or illustrated herein may represent portions of a single application. In addition, in some embodiments one or more of these steps may represent or correspond to one or more software applications or programs that, when executed by a computing device, may cause the computing device to perform one or more tasks, such as the method step.

In addition, one or more of the devices described herein may transform data, physical devices, and/or representations of physical devices from one form to another. Additionally or alternatively, one or more of the modules recited herein may transform a processor, volatile memory, non-volatile memory, and/or any other portion of a physical computing device from one form of computing device to another form of computing device by executing on the computing device, storing data on the computing device, and/or otherwise interacting with the computing device.

The term “computer-readable medium,” as used herein, generally refers to any form of device, carrier, or medium capable of storing or carrying computer-readable instructions. Examples of computer-readable media comprise, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

The processor as described herein can be configured to perform one or more steps of any method disclosed herein. Alternatively or in combination, the processor can be configured to combine one or more steps of one or more methods as disclosed herein.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising.” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for case of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

In general, any of the apparatuses and methods described herein should be understood to be inclusive, but all or a sub-set of the components and/or steps may alternatively be exclusive and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. A method of automatically identifying a feature from a video of a surgical procedure, the method comprising:
receiving, by a processor, a reference to be searched;
identifying one or more descriptors from the reference;
searching for a correlation between the one or more descriptors from the reference and clusters of one or more descriptors from a bulk video frame set, wherein the bulk video frame set comprises a plurality of sampled video frames from the video of the surgical procedure, wherein the clusters of one or more descriptors from the bulk video frame set have been clustered by the one or more descriptors from the bulk video frame set;
selecting one or more images from the bulk video frame set based on the correlation; and
outputting the one or more images.

2. The method of claim 1, wherein receiving the reference comprises receiving a reference image.

3. The method of claim 1, wherein receiving the reference comprises receiving a reference image of one or more of: an MRI scan image, an x-ray image, a video frame, a photograph, or a combination of any of these.

4. The method of claim 1, wherein searching for the correlation comprises searching using a machine-learning agent.

5. The method of claim 1, further comprising forming the bulk video frame set by sampling the video frames from the video of the surgical procedure.

6. The method of claim 1, wherein the plurality of sampled video frames from the video of the surgical procedure forming the bulk video frame set have been sampled at a frame rate of between 1 and 10 frames per second.

7. The method of claim 1, further comprising clustering the one or more descriptors from the bulk video frame set.

8. The method of claim 1, wherein outputting the one or more images further comprises modifying the video of a surgical procedure to indicate the reference.

9. The method of claim 1, wherein outputting further comprises displaying the one or more images.

10. The method of claim 1, wherein the clusters of one or more descriptors are hierarchical.

11. The method of claim 1, wherein identifying one or more descriptors from the reference comprises using inputs to a last layer of a neural network applied to the reference to identify fc7 descriptors.

12. The method of claim 1, wherein searching for the correlation comprises performing semantic searching.

13. The method of claim 1, wherein the bulk video frame set comprises sampled video frames from a portion of the video of the surgical procedure.

14. The method of claim 1, wherein the searching for the correlation comprises performing a semantic search.

15. The method of claim 1, further comprising identifying a surgical stage from the video of the surgical procedure.

16. A method of automatically identifying a feature from a video of a surgical procedure, the method comprising:
receiving, by a processor, a reference image to be searched;
identifying one or more descriptors from the reference image;
searching for a correlation between the one or more descriptors from the reference image and clusters of one or more descriptors from a bulk video frame set, wherein the bulk video frame set comprises a plurality of sampled video frames from the video of the surgical procedure that have each been translated into the one or more descriptors and clustered by the one or more descriptors from the bulk video frame set, further wherein the plurality of sampled video frames have been paired with a set of metadata;
selecting one or more images from the bulk video frame set based on the correlation; and
outputting the one or more images and their corresponding metadata for display.

17. A system comprising:
one or more processors;
a memory coupled to the one or more processors, the memory storing computer-program instructions, that, when executed by the one or more processors, perform a computer-implemented method of automatically identifying a feature from a video of a surgical procedure comprising:
receiving, by a processor, a reference to be searched;
identifying one or more descriptors from the reference;
searching for a correlation between the one or more descriptors from the reference and clusters of one or more descriptors from a bulk video frame set, wherein the bulk video frame set comprises a plurality of sampled video frames from the video of the surgical procedure, wherein the clusters of one or more descriptors from the bulk video frame set have been clustered by the one or more descriptors from the bulk video frame set;
selecting one or more images from the bulk video frame set based on the correlation; and
outputting the one or more images.

18. The system of claim 17, wherein receiving the reference comprises receiving a reference image.

19. The system of claim 17, wherein receiving the reference comprises receiving a reference image of one or more of: an MRI scan image, an x-ray image, a video frame, a photograph, or a combination of any of these.

20. The system of claim 17, wherein searching for the correlation comprises searching using a machine-learning agent.

21. The system of claim 17, wherein the computer-implemented method further comprises forming the bulk video frame set by sampling the video frames from the video of the surgical procedure.

22. The system of claim 17, wherein the plurality of sampled video frames from the video of the surgical procedure forming the bulk video frame set have been sampled at a frame rate of between 1 and 10 frames per second.

23. The system of claim 17, wherein the computer-implemented method further comprises clustering the one or more descriptors from the bulk video frame set.

24. The system of claim 17, wherein outputting the one or more images further comprises modifying the video of a surgical procedure to indicate the reference.

25. The system of claim 17, wherein outputting further comprises displaying the one or more images.

26. The system of claim 17, wherein the clusters of one or more descriptors are hierarchical.

27. The system of claim 17, wherein identifying one or more descriptors from the reference comprises using inputs to a last layer of a neural network applied to the reference to identify fc7 descriptors.

28. The system of claim 17, wherein searching for the correlation comprises performing semantic searching.

29. The system of claim 17, wherein the bulk video frame set comprises sampled video frames from a portion of the video of the surgical procedure.

30. The system of claim 17, wherein the searching for the correlation comprises performing a semantic search.

31. The system of claim 17, wherein the computer-implemented method further comprises identifying a surgical stage from the video of the surgical procedure.

32.-51. (canceled)
